(19) United States
(12) Patent Application Publication
(10) Pub. No.: US 2007/0218145 A1
(43) Pub. Date: Sep. 20, 2007

(54) METHODS AND KITS FOR POTENTIATING SKIN CARE AGENTS

(76) Inventor: Dennis F. Gross, New York, NY (US)

Correspondence Address: KENYON & KENYON LLP, 1500 K STREET N.W., SUITE 700, WASHINGTON, DC, 20005, US

(21) Appl. No.: 11/686,690

(22) Filed: Mar. 15, 2007

Related U.S. Application Data
(60) Provisional application No. 60/783,069, filed on Mar. 17, 2006.

(51) Int. Cl. A61K 33/06

(52) U.S. Cl. 424/684

(57) ABSTRACT

The present invention provides a treatment method for administering a skin care product to the skin of a subject comprising applying a self heat generation composition into an area of the skin to generate heat locally, wherein the skin care product is administered to the area of the skin before, along with, or after the application of the self heat generation composition. The self heat generation composition comprises at least one physiologically acceptable silicoaluminate, preferably sodium silicoaluminate, with the optional inclusion of at least one skin care agent, and wherein the self heat generation composition is preferably massaged into the area of the skin to promote heat generation. The present invention also provides a kit useful for skin treatment, wherein the kit comprises a skin care product and a self heat generation composition, wherein the skin care product comprises at least one skin care agent and the self heat generation composition comprises at least one physiologically acceptable silicoaluminate.

### CROSS REFERENCE TO RELATED APPLICATION

The present application claims the benefit of U.S. Provisional Application No. 60/783,069 filed Mar. 17, 2006, the disclosures of which are hereby incorporated by reference.

The present invention is related to methods and kits for enhancing the effects of skin care agents administered topically to a subject involving self heat generation.

### BACKGROUND INFORMATION

Skin care products have been widely used by consumers and professionals alike in achieving healthy and attractive skin. The present invention aims at improving the effects of skin care agents administered topically by applying the skin care agents with a self heat generation composition.

### SUMMARY OF THE INVENTION

The present invention provides a skin treatment method for administering a skin care agent to the skin of a subject comprising applying a self heat generation composition into an area of the skin to generate heat locally, wherein the skin care agent is administered to the area of the skin before, along with, or after the application of the self heat generation composition. When the skin care agent is administered to the area of the skin before the application of the self heat generation composition, the skin care agent is applied, preferably, within 1 hour, more preferably, within 30 minutes, further more preferably, within 15 minutes, even more preferably, within 10 minutes, much more preferably, within 5 minutes and most preferably, within 1 minute, of the application of the self heat generation composition. When the skin care agent is administered to the area of the skin after the application of the self heat generation composition, the skin care agent is applied, preferably, within 20 minutes, more preferably, within 15 minutes, further more preferably, within 10 minutes, even more preferably, within 5 minutes, much more preferably, within 2 minutes and most preferably, within 1 minute, of the application of the self heat generation composition. The self heat generation composition comprises at least one physiologically acceptable silicoaluminate, preferably sodium silicoaluminate, and wherein the self heat generation composition is preferably massaged into the area of the skin to promote heat generation. In some of the embodiments of the skin treatment method of the present invention, the skin treatment method does not comprise the application of hyaluronic acid to the skin.

Some of the embodiments of the skin treatment method of the present invention further comprises, after the application of the self heat generation composition,

(b) optionally letting the self heat generation composition set on the area of the skin to form a mask;

(c) optionally massaging the mask into the area of the skin; and

(d) optionally removing the mask from the area of the skin.

After and/or before the skin treatment method, another skin care agent can be optionally applied to the area of the skin. The skin care agent optionally applied before and/or after the treatment method can be, but is not limited to, a moisturizer, a skin peel product such as an acid/alkaline skin peel product disclosed in U.S. patent application Ser. No. 09/338,729 filed on Jun. 23, 1999 (the disclosure of which application is hereby incorporated by reference), Antioxidant Firming Face Serum and/or Anti-Aging Vitamin C Gel.

The present invention also provides a kit useful for skin treatment or administering a skin care agent to the skin, wherein the kit comprises a skin care product and a self heat generation composition, and wherein the skin care product comprises at least one skin care agent and the self heat generation composition comprises a physiologically acceptable silicoaluminate, preferably sodium silicoaluminate. Preferably, the kit further comprises at least one device, e.g., brush or pad, for applying the skin care product and/or the self heat generation composition to the skin. The kit optionally further comprises an instruction for applying the skin care product and self heating generation composition to the skin. The instruction preferably describes applying the skin care product and self heating generation composition to the skin for promoting or enhancing the beneficial dermal effect(s) of the skin care agent. In some of the embodiments of the kit of the present invention, the kit does not comprise hyaluronic acid.

In addition, the present invention provides a skin care composition comprising a physiologically acceptable silicoaluminate, preferably sodium silicoaluminate, and at least one skin care agent.

The method and kit of the present invention are useful in allowing consumers to achieve professional skin care results at home in a small number of easy steps. The skin care agents can plump trouble areas minimizing fine lines and wrinkles caused by dehydration. The self heat generation composition can create a barrier on the skin increasing circulation and enhancing the dermal absorption of the skin care agents, optionally forcing the skin care agents deep into the skin to achieve more beneficial effects such as scar reduction and/or skin firming from the skin care agents, e.g., antioxidants, collagen simulating ingredients, collage promoters, soluble collagens, self tanners. Skin treatment using the method and/or kit of the invention can leave dry, dehydrated skin immediately plumper, smoother and younger-looking.

Before and/or after the application of the method or kit of the present invention to the skin, the skin can optionally be subjected to one or more skin care treatments, e.g., facial, acne treatment, acne prevention, and skin peel, performed by a professional or consumer. For instance, the one or more skin care treatments can be performed to the skin within 12 hours, preferably within 6 hours, more preferably within 3 hours, further more preferably within 1 hour, even more preferably within 30 minutes, and most preferably within 15 minutes, before and/or after the application of the method or kit of the present invention to the skin.

The method and kit of the present invention are useful in enhancing or potentiating the effects of the skin care agent, and/or enhancing the dermal absorption of the skin care agent.

### DETAILED DESCRIPTION OF THE INVENTION

In the present application, the term “subject” means a mammal, preferably a human. The subject can be a human consumer or patient. More preferably, the subject is a human consumer.

The term “physiologically acceptable” modifying a substance means that the substance would cause no significant adverse health effect when administered to the subject. Preferably, a “physiologically acceptable” substance would cause little or no allergic response.

As used herein, the term “cosmetically acceptable” modifying a substance means that the substance is of sufficiently high purity and suitable for use in contact with human skin without undue toxicity, incompatibility and instability. A “cosmetically acceptable” substance, preferably, causes little or no allergic response.

The term “skin care agent” refers to an agent that has one or more beneficial effects on the care and/or hygiene of the skin. The skin care agent can be selected from the group consisting of antioxidants, free-radical scavengers, antimicrobial agents, anti-acne agents, reducing agents, vitamins, skin protecting agents, skin bleaching agents, skin conditioning agents, skin soothing agents, skin healing agents, collagen promoters, exfoliators, self tanners, chelators, hair removers, anti-erythema agents, anti-redness agents, anti-rosacea agents, depuffing agents, anti-edema agents, anti-swelling agents, green tea extract, P. emblica (Amla), arnica, chamomile extract and cucumber extract. The skin care agent is, preferably, selected from the group consisting of skin protecting agents, skin conditioning agents, antioxidants, anti-acne agents, collagen promoters, soluble collagen, self tanner and mixtures thereof. More preferably, the skin care agent is selected from antiwrinkle agents, anti-skin-atrophy agents, antioxidants and anti-acne agents. In some of the embodiments of the present invention, the skin care agent is not hyaluronic acid or the skin care agent does not comprise hyaluronic acid.

The self heat generation composition comprises at least one physiologically acceptable silicoaluminate. Examples of “physiologically acceptable silicoaluminate” include sodium silicoaluminate, potassium silicoaluminate, calcium silicoaluminate, magnesium silicoaluminate and zinc silicoaluminate. Optionally, the self heat generation composition further comprises at least one skin care agent. In some of the embodiments of the present invention, the self heat generation composition does not comprise hyaluronic acid.

The self heat generation composition, preferably, further comprises at least one emollient/humectant/moisturizer (e.g., butylene glycol). The self heat generation composition may further comprise at least one additional active ingredient, at least one cosmetically acceptable aesthetic component, at least one cosmetically acceptable excipient and/or at least one cosmetically acceptable vehicle or carrier.

The at least one cosmetically acceptable vehicle or carrier is, preferably, water or a cosmetically acceptable aqueous buffer having a pH of about 7.0 to about 7.4, and, more preferably, the water or aqueous buffer is purified and/or sterile.

The at least one cosmetically acceptable excipient is selected from the group consisting of surfactant/emulsifying agents, absorbents, antifoaming agents, binders, biological additives, chelating agents, denaturants, preservatives, solubilizing agents, solvents and thickening agents.

The at least one additional active ingredient is selected from the group consisting of antioxidants, free-radical scavengers, antimicrobial agents, topical analgesics, steroidal anti-inflammatory drugs, anti-acne agents, reducing agents, vitamins, skin protecting agents, skin bleaching agents, skin conditioning agents, skin soothing agents, skin healing agents, collagen promoters, exfoliators, self tanners, chelators, hair removers, anti-erythema agents, anti-redness agents, anti-rosacea agents, depuffing agents, anti-edema agents, anti-swelling agents, green tea extract, P. emblica (Amla), arnica, chamomile extract and cucumber extract. The at least one active ingredient is, preferably, at least one antioxidant, anti-acne agent, collagen promoter, soluble collagen, self tanner or mixtures thereof. More preferably, the at least one additional active ingredient is at least one antioxidant or anti-acne agent.

Suitable collagen promoters are peptides.

Suitable surfactant/emulsifying agents include ceteareths, ceteths, laneths, laureths, isoseareths, steareths, cetyl alcohol, deceths, dodoxynols, glyceryl palmitate, glyceryl stearate, laneths, myreths, nonoxynols, octoxynols, oleths, PEG-castor oil, poloxamers (e.g., poloxamer 407), poloxamines, polysorbates, sodium laurate, ammonium laureth sulfate, sodium laureth sulfate, sodium lauroyl sarcosinate, sodium lauroyl taurate, sodium lauryl sulfate, sodium methyl cocoyl taurate, sodium methyl oleoyl taurate, sodium nonoxynol sulfate, sodium cetyl sulfate, sodium cetearyl sulfate, sodium cocoate, sodium cocoyl isethionate and sodium cocoyl sarcosinate. Other suitable surfactant/emulsifying agents would be known to one of skill in the art and are listed in the CTFA International Cosmetic Ingredient Dictionary and Handbook, Vol. 2, 7th Edition (1997). Preferred surfactants include octoxynol-9 and polysorbate-20.

Examples of chelating agents are disodium EDTA, trisodium EDTA, tetrasodium EDTA and sodium metasilicate.

Examples of suitable preservatives include imidazolidinyl urea, diazolidinyl urea, phenoxyethanol, methylparaben, ethylparaben and propylparaben.

Examples of thickening agents include isopropyl myristate, isopropyl palmitate, isodecyl neopentanoate, squalene, mineral oil, C₁₂-C₁₅ benzoate and hydrogenated polyisobutene.

Examples of antioxidants and free-radical scavengers include ascorbic acid, salts of ascorbic acid such as ascorbyl palmitate and sodium ascorbate, ascorbyl glucosamine, vitamin E (i.e., tocopherols such as α-tocopherol), derivatives of vitamin E (e.g., tocopheryl acetate), retinoids such as retinoic acid, retinol, trans-retinol, cis-retinol, mixtures of trans-retinol and cis-retinol, 3-dehydroretinol and derivatives of vitamin A (e.g., retinyl acetate, retinal and retinyl palmitate, also known as tetinyl palmitate), sodium citrate, sodium sulfite, lycopene, anthocyanids, bioflavinoids (e.g., hesperitin, naringen, rutin and quercetin), superoxide dismutase, glutathione peroxidase, butylated hydroxytoluene (BHT), butylated hydroxyanisole (BHA), indole-3-carbinol, pycnogenol, melatonin, sulforaphane, pregnenolone, lipoic acid and 4-hydroxy-5-methyl-3 [2H]-furanone.

The antimicrobial agents can be antibacterial agents and/or antifungal agents. Examples of the antimicrobial agents include benzoyl peroxide, erythromycin, tetracycline, triclosan, azelaic acid, clindamycin, chlorhexidine, neomycin, miconazole and clotrimazole.

Examples of topical analgesics include aspirin and non-steroidal anti-inflammatory drugs. Suitable non-steroidal anti-inflammatory drugs include ibuprofen, naproxen, benoxaprofen, flurbioprofen, fenoprofen, fenbufen, ketoprofen, indoprofen, pirprofen, carprofen, oxaprozin, pranoprofen, microprofen, tioxaprofen, suprofen, alminoprofen, tiaprofenic acid, fluprofen and bucloxic acid.

The skin conditioning agents can be emollients, humectants and moisturizers, which include urea; guanidine; aloe vera; glycolic acid and glycolate salts such as ammonium and quaternary alkyl ammonium; lactic acid and lactate salts such as sodium lactate, ammonium lactate and quaternary alkyl ammonium lactate; polyhydroxy alcohols such as sorbitol, glycerol, hexanetriol, propylene glycol, butylene glycol, hexylene glycol, polyethylene glycol; carbohydrates such as alkoxylated glucose; starches; starch derivatives; glycerin; pyrrolidone carboxylic acid (PCA); lactamide monoethanolamine; acetamide monoethanolamine; volatile silicone oils; nonvolatile silicone oils; and mixtures thereof. Suitable silicone oils can be polydialkylsiloxanes, polydiarylsiloxanes, polyalkarylsiloxanes and cyclomethicones having 3 to 9 silicon atoms.

Skin soothing agents include bisabolol.

Suitable steroidal anti-inflammatory drugs include, for example, hydrocortisone and bensonide.

Suitable anti-acne agents can be drying agents, keratolytic agents, epidermolytic agents, antimicrobial agents and retinoids. Examples of anti-acne agents include sulfur, resorcinol, glycolic acid, lactic acid, pyruvic acid, salicylic acid, retinoic acid, derivatives of retinoic acid, and antimicrobial agents, e.g., benzoyl peroxide, erythromycin, triclosan, azelaic acid, clindamycin, chlorhexidine, neomycin, miconazole, clotrimazole and tetracycline.

The skin protecting agents are agents that protect the skin against chemical irritants and/or physical irritants, e.g., UV light, including sunscreens, anti-acne additives, anti-wrinkle and anti-skin atrophy agents. Suitable sunscreens as skin protecting agents include 2-ethylhexyl p-methoxycinnamate, 2-ethylhexyl N,N-dimethyl-p-aminobenzoate, p-aminobenzoic acid, 2-phenylbenzimidazole-5-sulfonic acid, octocrylene, oxybenzone, homomenthyl salicylate, octyl salicylate, 4,4′-methoxy-t-butyldibenzoylmethane, 4-isopropy dibenzoylmethane, 3-benzylidene camphor, 3-(4-methylbenzylidene) camphor, anthanilates, ultrafine titanium dioxide, zinc oxide, iron oxide, silica, 4-N,N-(2-ethylhexyl)methylaminobenzoic acid ester of 2,4-dihydroxybenzophenone, 4-N,N-(2-ethylhexyl)-methylaminobenzoic acid ester with 4-hydroxydibenzoylmethane, 4-N,N-(2-ethylhexyl)-methylaminobenzoic acid ester of 2-hydroxy-4-(2-hydroxyethoxy)benzophenone and 4-N,N(2-ethylhexyl)-methylaminobenzoic acid ester of 4-(2-hydroxyethoxy)dibenzoylmethane. Suitable anti-acne agents include salicylic acid; 5-octanoyl salicylic acid; resorcinol; retinoids such as retinoic acid and its derivatives; sulfur-containing D and L amino acids other than cysteine; lipoic acid; antibiotics and antimicrobials such as benzoyl peroxide, octopirox, tetracycline, 2,4,4′-trichloro-2′-hydroxydiphenyl ether, 3,4,4′-trichlorobanilide, azelaic acid, phenoxyethanol, phenoxypropanol, phenoxisopropanol, ethyl acetate, clindamycin and melclocycline; flavonoids; and bile salts such as scymnol sulfate, deoxycholate and cholate. Examples of anti-wrinkle and anti-skin atrophy agents are retinoic acid and its derivatives, retinol, retinyl esters, salicylic acid and its derivatives, sulfur-containing D and L amino acids except cysteine, alpha-hydroxy acids (e.g., glycolic acid and lactic acid), phytic acid, lipoic acid and lysophosphatidic acid.

Suitable skin bleaching agents include, for example, hydroquinone, kojic acid and sodium metabisulfite.

The at least one aesthetic agent can be at least one of fragrances, pigments, colorants, essential oils, skin sensates and astringents. Suitable aesthetic agents include clove oil, menthol, camphor, eucalyptus oil, eugenol, methyl lactate, bisabolol, witch hazel distillate (preferred) and green tea extract (preferred).

The at least one anti-oxidant preferably is selected from ascorbic acid, ascorbyl palmitate, ascorbyl glucosamine, tocopheryl acetate, retinyl palmitate, superoxide dismutase, or mixtures thereof. The at least one skin conditioning agent preferably is selected from cyclomethicone and/or dimethiconol. The at least one reducing agent preferably is ubiquinone. The at least one additional active agent can be Camellia sinensis leaf extract and/or white tea extract or juice, preferably Camellia sinensis leaf extract. The at least one cosmetically acceptable vehicle or carrier can be purified or sterile water, preferably purified water. The at least one cosmetically acceptable excipient can be phospholipids.

In one of the embodiments of the present invention, the skin care product comprises cyclomethicone, dimethiconol, sodium hyaluronate, Camellia sinensis leaf extract, ascorbyl palmitate, ascorbyl glucosamine, tocopheryl acetate, retinyl palmitate, superoxide dismutase, ubiquinone, phospholipids and purified water with the optional inclusion of ascorbic acid.

In some of the embodiments of the present invention, the self heat generation composition comprises at least one physiologically acceptable silicoaluminate, preferably sodium silicoaluminate, at least one skin conditioning agent, at least one surfactant, at least one additional active agent, at least one anti-oxidant, at least one cosmetically acceptable excipient and at least one aesthetic agent, and optionally at least one cosmetically acceptable vehicle or carrier. The at least one skin conditioning agent can be butylene glycol and/or petrolatum. The at least one surfactant can be PEG-8 and/or methyl GLUCETH-20. The at least one additional active agent can be Camellia sinensis leaf extract and/or white tea extract or juice. The at least one anti-oxidant can be ascorbyl palmitate, retinyl palmitate, tocopheryl acetate, or mixtures thereof. The at least one cosmetically acceptable excipient can be hydroxypropylcellulose and/or hydroxypropyl methylcellulose.

In one of the embodiments of the present invention, the self heat generation composition comprises sodium silicoaluminate, butylene glycol, PEG-8, Camellia sinensis leaf extract, white tea extract or juice, ascorbyl palmitate, retinyl palmitate, tocopheryl acetate, dimethicone, methyl GLUCETH-20, hydroxypropylcellulose, hydroxypropyl methylcellulose, petrolatum and titanium dioxide, with optional inclusion of phospholipids.

In one of the embodiments of the present invention, the self heat generation composition comprises the following ingredients:

| Ingredient | Amount (weight %) |
|---|---|
| sodium silicoaluminate | about 2% to about 99%, |
| butylene glycol | about 0% to about 98%, |
| PEG-8 | about 0% to about 25%, |
| Camellia sinensis leaf extract | about 0% to about 20%, |
| white tea extract or juice | about 0% to about 20%, |
| ascorbyl palmitate | about 0% to about 20%, |
| retinyl palmitate | about 0% to about 20%, |
| tocopheryl acetate | about 0% to about 20%, |
| dimethicone | about 0% to about 20%, |
| methyl GLUCETH-20 | about 0% to about 20%, |
| hydroxypropylcellulose | about 0% to about 20%, |
| hydroxypropyl methylcellulose | about 0% to about 20%, |
| petrolatum | about 0% to about 20%, |
| titanium dioxide | about 0% to about 20% and |
| optional phospholipids | about 0% to about 20%. |

In the embodiment, the self heat generation composition preferably comprises the following ingredients:

| Ingredient | Preferred Amount (Weight %) |
|---|---|
| sodium silicoaluminate | about 10% to about 80%, |
| butylene glycol | about 10% to about 80%, |
| PEG-8 | about 0.1% to about 10%, |
| Camellia sinensis leaf extract | about 0.01% to about 10%, |
| white tea extract or juice | about 0.01% to about 10%, |
| ascorbyl palmitate | about 0.01% to about 10%, |
| retinyl palmitate | about 0.01% to about 10%, |
| tocopheryl acetate | about 0.01% to about 10%, |
| dimethicone | about 0.01% to about 10%, |
| methyl GLUCETH-20 | about 0.01% to about 10%, |
| hydroxypropylcellulose | about 0.01% to about 10%, |
| hydroxypropyl methylcellulose | about 0.01% to about 10%, |
| petrolatum | about 0.01% to about 10%, |
| titanium dioxide | about 0.01% to about 10% and |
| optional phospholipids | about 0.01% to about 10% (if present). |

In the embodiment, the self heat generation composition more preferably comprises the following ingredients:

| Ingredient | More Preferred Amount (Weight %) |
|---|---|
| sodium silicoaluminate | about 20% to about 70%, |
| butylene glycol | about 20% to about 70%, |
| PEG-8 | about 0.5% to about 5%, |
| Camellia sinensis leaf extract | about 0.01% to about 2%, |
| white tea extract or juice | about 0.01% to about 2%, |
| ascorbyl palmitate | about 0.01% to about 2%, |
| retinyl palmitate | about 0.01% to about 2%, |
| tocopheryl acetate | about 0.01% to about 2%, |
| dimethicone | about 0.01% to about 2%, |
| methyl GLUCETH-20 | about 0.01% to about 2%, |
| hydroxypropylcellulose | about 0.01% to about 2%, |
| hydroxypropyl methylcellulose | about 0.01% to about 2%, |
| petrolatum | about 0.5% to about 5%, |
| titanium dioxide | about 0.01% to about 2% and |
| optional phospholipids | about 0.01% to about 2% (if present). |

In the embodiment, the self heat generation composition even more preferably comprises the following ingredients:

| Ingredient | Amount (Weight %) |
|---|---|
| sodium silicoaluminate | about 31.84%, |
| butylene glycol | about 62.02%, |
| PEG-8 | about 3.35%, |
| Camellia sinensis leaf extract | about 0.10%, |
| white tea extract or juice | about 0.01%, |
| ascorbyl palmitate | about 0.01%, |
| retinyl palmitate | about 0.01%, |
| tocopheryl acetate | about 0.01%, |
| dimethicone | about 0.42%, |
| methyl GLUCETH-20 | about 42%, |
| hydroxypropylcellulose | about 0.30%, |
| hydroxypropyl methylcellulose | about 0.30%, |
| petrolatum | about 1%, |
| titanium dioxide | about 0.20% and |
| optional phospholipids | about 0.01% (if present). |

In some of the embodiments of the method of the present invention, the self heat generation composition is preferably massaged into the skin with damp fingers to promote heat generation. Preferably, the self heat generation composition is massaged into the skin for about 20 seconds to about 3 minutes, more preferably about 0.5 minute to about 2 minutes, and even more preferably about 1 minute, to activate heat.

In some of the embodiments of the method of the present invention, the self heat generation composition is set on the area of the skin in step (b) for preferably about 3 minutes to about 30 minutes, more preferably about 5 minutes to about 20 minutes, and even more preferably about 10 minutes to about 15 minutes. Without being bound by any theory, it is believed that the setting of the self-heating mask would provide heat and moisture to the area of the skin.

In some of embodiments of the method of the present invention, the optional massaging in step (c) is performed for about 20 seconds to about 3 minutes, preferably about 0.5 minute to about 2 minutes, and more preferably about 1 minute.

In some of embodiments of the method of the present invention, the mask is removed from the area of the skin in step (d) preferably with a warm towel, wherein the warm towel may optionally be wet.

In some of embodiments of the method of the present invention, the optional additional skin care product applied before and/or after the treatment method of the invention can be a skin peeling product, e.g., the skin peeling product disclosed in U.S. patent application Ser. No. 09/338,729, filed Jun. 23, 1999, the disclosure of which is incorporated by reference herein.

The present invention also provides 27 embodiments of a kit for administering at least one skin care agent to the skin of a human subject for treating the skin as listed below.

### 1. A kit for administering at least one skin care agent to the skin of a human subject comprising

(a) at least one skin care agent; and

(b) a self heat generation composition that can generate heat locally when applied to the skin, wherein the self heat generation composition comprises at least one physiologically acceptable silicoaluminate.

### 2. The kit of kit embodiment 1, wherein the silicoaluminate is sodium silicoaluminate, potassium silicoaluminate, lithium silicoaluminate, calcium silicoaluminate, magnesium silicoaluminate or zinc silicoaluminate.

### 3. The kit of kit embodiment 2, wherein the silicoaluminate is sodium silicoaluminate.

### 4. The kit of kit embodiment 1, wherein the at least one skin care agent is selected from anti-acne agents, skin firming promoters, collagen promoters, soluble collagen and self tanners.

### 5. The kit of kit embodiment 4, wherein the at least one skin care agent comprises a skin firming promoter.

### 6. The kit of kit embodiment 4, wherein the at least one skin care agent comprises a collagen promoter.

### 7. The kit of kit embodiment 4, wherein the at least one skin care agent comprises an anti-acne agent.

### 8. The kit of kit embodiment 4, wherein the at least one skin care agent comprises a self tanner.

### 9. The kit of kit embodiment 1, wherein the self heat generation composition further comprises at least one cosmetically acceptable excipient, and/or at least one cosmetically acceptable vehicle or carrier.

### 10. The kit of kit embodiment 9, wherein the at least one cosmetically acceptable vehicle or carrier is water or a cosmetically acceptable aqueous buffer having a pH of about 7.0 to about 7.4.

### 11. The kit of kit embodiment 10, wherein the water or aqueous buffer is purified and/or sterile.

12. The kit of kit embodiment 9, wherein the at least one cosmetically acceptable excipient is selected from the group consisting of surfactant/emulsifying agents, absorbents, antifoaming agents, binders, biological additives, chelating agents, denaturants, preservatives, solubilizing agents, solvents and thickening agents.

### 13. The kit of kit embodiment 12, wherein the at least one cosmetically acceptable excipient comprises at least one surfactant/emulsifying agent.

### 14. The kit of kit embodiment 1, wherein the self heat generation composition further comprises at least one additional active ingredient.

15. The kit of kit embodiment 14, wherein the at least one additional active ingredient is selected from the group consisting of antioxidants, free-radical scavengers, antimicrobial agents, topical analgesics, steroidal anti-inflammatory drugs, anti-acne agents, reducing agents, vitamins, skin protecting agents, skin bleaching agents, skin conditioning agents, skin soothing agents, skin healing agents, green tea extract, P. emblica (Amla), amica, chamomile extract and cucumber extract.

### 16. The kit of kit embodiment 15, wherein the at least one additional active ingredient comprises at least one antioxidant.

17. The kit of kit embodiment 1, wherein the at least one skin care agent is present in a skin care product comprising the following ingredients:

| Ingredient | Amount (Weight %) |
|---|---|
| cyclomethicone | about 0% to about 99%, |
| dimethiconol | about 0% to about 30%, |
| sodium hyaluronate | about 0.005% to about 99.5%, |
| Camellia sinensis leaf extract | about 0% to about 20%, |
| optional ascorbic acid | about 0% to about 20%, |
| ascorbyl palmitate | about 0% to about 20%, |
| ascorbyl glucosamine | about 0% to about 20%, |
| tocopheryl acetate | about 0% to about 20%, |
| retinyl palmitate | about 0% to about 20%, |
| superoxide dismutase | about 0% to about 1%, |
| ubiquinone | about 0% to about 20%, |
| phospholipids | about 0% to about 20% and |
| purified water | about 0% to about 50%. |

18. The kit of kit embodiment 17, wherein the skin care product comprises the following ingredients:

| Ingredient | Amount (Weight %) |
|---|---|
| cyclomethicone | about 5% to about 95%, |
| dimethiconol | about 5% to about 25%, |
| sodium hyaluronate | about 0.01% to about 85%, |
| Camellia sinensis leaf extract | about 0.01% to about 10%, |
| optional ascorbic acid | about 0.01% to about 10% (if present), |
| ascorbyl palmitate | about 0.01% to about 10%, |
| ascorbyl glucosamine | about 0.01% to about 10%, |
| tocopheryl acetate | about 0.01% to about 10%, |
| retinyl palmitate | about 0.01% to about 10%, |
| superoxide dismutase | about 0.0001% to about 0.1%, |
| ubiquinone | about 0.01% to about 10%, |
| phospholipids | about 0.01% to about 10% and |
| purified water | about 0.01% to about 20%. |

19. The kit of kit embodiment 18, wherein the skin care product comprises the following ingredients:

| Ingredient | Amount (Weight %) |
|---|---|
| cyclomethicone | about 10% to about 90%, |
| dimethiconol | about 10% to about 20%, |
| sodium hyaluronate | about 0.01% to about 70%, |
| Camellia sinensis leaf extract | about 0.01% to about 5%, |
| optional ascorbic acid | about 0.01% to about 5% (if present), |
| ascorbyl palmitate | about 0.01% to about 5%, |
| ascorbyl glucosamine | about 0.01% to about 5%, |
| tocopheryl acetate | about 0.01% to about 5%, |
| retinyl palmitate | about 0.01% to about 5%, |
| superoxide dismutase | about 0.0001% to about 0.01%, |
| ubiquinone | about 0.01% to about 5%, |
| phospholipids | about 0.01% to about 5% and |
| purified water | about 0.01% to about 10%. |

20. The kit of kit embodiment 18, wherein the skin care product comprises the following ingredients:

| Ingredient | Amount (Weight %) |
|---|---|
| cyclomethicone | about 86.90%, |
| dimethiconol | about 13.00%, |
| sodium hyaluronate | about 0.01%, |
| Camellia sinensis leaf extract | about 0.01%, |
| optional ascorbic acid | about 0.01% (if present), |
| ascorbyl palmitate | about 0.01%, |
| ascorbyl glucosamine | about 0.01%, |
| tocopheryl acetate | about 0.01%, |
| retinyl palmitate | about 0.01%, |
| superoxide dismutase | about 0.0001%, |
| ubiquinone | about 0.02%, |
| phospholipids | about 0.01% and |
| purified water | about 0.01%. |

21. The kit of kit embodiment 1, wherein the self heat generation composition comprises sodium silicoaluminate, butylene glycol, PEG-8, Camellia sinensis leaf extract, white tea extract or juice, ascorbyl palmitate, retinyl palmitate, tocopheryl acetate, dimethicone, methyl GLUCETH-20, hydroxypropylcellulose, hydroxypropyl methylcellulose, petrolatum and titanium dioxide, with optional inclusion of phospholipids.
22. The kit of kit embodiment 1, wherein the self heat generation composition comprises the following ingredients:

| Ingredient | Amount (weight %) |
|---|---|
| sodium silicoaluminate | about 2% to about 99%, |
| butylene glycol | about 0% to about 98%, |
| PEG-8 | about 0% to about 25%, |
| Camellia sinensis leaf extract | about 0% to about 20%, |
| white tea extract or juice | about 0% to about 20%, |
| ascorbyl palmitate | about 0% to about 20%, |
| retinyl palmitate | about 0% to about 20%, |
| tocopheryl acetate | about 0% to about 20%, |
| dimethicone | about 0% to about 20%, |
| methyl GLUCETH-20 | about 0% to about 20%, |
| hydroxypropylcellulose | about 0% to about 20%, |
| hydroxypropyl methylcellulose | about 0% to about 20%, |
| petrolatum | about 0% to about 20%, |
| titanium dioxide | about 0% to about 20% and |
| optional phospholipids | about 0% to about 20%. |

23. The kit of kit embodiment 22, wherein the self heat generation composition comprises the following ingredients:

| Ingredient | Preferred Amount (Weight %) |
|---|---|
| sodium silicoaluminate | about 10% to about 80%, |
| butylene glycol | about 10% to about 80%, |
| PEG-8 | about 0.1% to about 10%, |
| Camellia sinensis leaf extract | about 0.01% to about 10%, |
| white tea extract or juice | about 0.01% to about 10%, |
| ascorbyl palmitate | about 0.01% to about 10%, |
| retinyl palmitate | about 0.01% to about 10%, |
| tocopheryl acetate | about 0.01% to about 10%, |
| dimethicone | about 0.01% to about 10%, |
| methyl GLUCETH-20 | about 0.01% to about 10%, |
| hydroxypropylcellulose | about 0.01% to about 10%, |
| hydroxypropyl methylcellulose | about 0.01% to about 10%, |
| petrolatum | about 0.1% to about 10%, |
| titanium dioxide | about 0.01% to about 10% and |
| optional phospholipids | about 0.01% to about 10% (if present). |

24. The kit of kit embodiment 23, wherein the self heat generation composition comprises the following ingredients:

| Ingredient | More Preferred Amount (Weight %) |
|---|---|
| sodium silicoaluminate | about 20% to about 70%, |
| butylene glycol | about 20% to about 70%, |
| PEG-8 | about 0.5% to about 5%, |
| Camellia sinensis leaf extract | about 0.01% to about 2%, |
| white tea extract or juice | about 0.01% to about 2%, |
| ascorbyl palmitate | about 0.01% to about 2%, |
| retinyl palmitate | about 0.01% to about 2%, |
| tocopheryl acetate | about 0.01% to about 2%, |
| dimethicone | about 0.01% to about 2%, |
| methyl GLUCETH-20 | about 0.01% to about 2%, |
| hydroxypropylcellulose | about 0.01% to about 2%, |
| hydroxypropyl methylcellulose | about 0.01% to about 2%, |
| petrolatum | about 0.5% to about 5%, |
| titanium dioxide | about 0.01% to about 2% and |
| optional phospholipids | about 0.01% to about 2% (if present). |

25. The kit of kit embodiment 24, wherein the self heat generation composition comprises the following ingredients:

| Ingredient | Amount (Weight %) |
|---|---|
| sodium silicoaluminate | about 31.84%, |
| butylene glycol | about 62.02%, |
| PEG-8 | about 3.35%, |
| Camellia sinensis leaf extract | about 0.10%, |
| white tea extract or juice | about 0.01%, |
| ascorbyl palmitate | about 0.01%, |
| retinyl palmitate | about 0.01%, |
| tocopheryl acetate | about 0.01%, |
| dimethicone | about 0.42%, |
| methyl GLUCETH-20 | about 42%, |
| hydroxypropylcellulose | about 0.30%, |
| hydroxypropyl methylcellulose | about 0.30%, |
| petrolatum | about 1%, |
| titanium dioxide | about 0.20% and |
| optional phospholipids | about 0.01% (if present). |

### 26. The kit of kit embodiment 1, further comprising an applicator for applying the skin care agent and/or self heat generation composition to the skin.

27. The kit of kit embodiment 26, wherein the applicator is a brush or absorbent pad.

What is claimed is:

1. A skin treatment method for administering a skin care agent to the skin of a subject comprising
applying a self heat generation composition into an area of the skin of a subject to generate heat locally; and
applying the skin care agent to the area of the skin before, along with, or after the application of the self heat generation composition,
wherein the self heat generation composition comprises at least one physiologically acceptable silicoaluminate.

2. The skin treatment method of claim 1, further comprising letting the self heat generation composition set on the area of the skin to form a mask.

3. The skin treatment method of claim 2, further comprising massaging the mask into the area of the skin.

4. The skin treatment method of claim 3, further comprising removing the mask from the area of the skin.

5. The skin treatment method of claim 1, wherein the at least one physiologically acceptable silicoaluminate is selected from the group consisting of sodium silicoaluminate, potassium silicoaluminate, calcium silicoaluminate, magnesium silicoaluminate and zinc silicoaluminate.

6. The skin treatment method of claim 5, wherein the at least one physiologically acceptable silicoaluminate is sodium silicoaluminate.

7. The skin treatment method of claim 1, wherein the self heat generation composition further comprises at least one emollient/humectant/moisturizer.

8. The skin treatment method of claim 1, wherein the self heat generation composition further comprises at least one additional active ingredient.

9. The skin treatment method of claim 8, wherein the at least one additional active ingredient is selected from the group consisting of antioxidants, free-radical scavengers, antimicrobial agents, topical analgesics, steroidal anti-inflammatory drugs, anti-acne agents, reducing agents, vitamins, skin protecting agents, skin bleaching agents, skin conditioning agents, skin soothing agents, skin healing agents, green tea extract, P. emblica (Amla), amica, chamomile extract and cucumber extract.

10. The skin treatment method of claim 9, wherein the at least one additional active ingredient is selected from the group consisting of antioxidants and anti-acne agents.

11. The skin treatment method of claim 1, wherein the skin care agent is selected from the group consisting of anti-acne agents, skin firming promoters, antioxidants, collagen stimulating ingredients, collagen promoters, soluble collagen and self tanners.

12. The skin treatment method of claim 1, wherein the self heat generation composition further comprises at least one cosmetically acceptable aesthetic component.

13. The skin treatment method of claim 1, wherein the self heat generation composition further comprises at least one cosmetically acceptable excipient and/or at least one cosmetically acceptable vehicle or carrier.

14. The skin treatment method of claim 13, wherein the at least one cosmetically acceptable vehicle or carrier is selected from water and a cosmetically acceptable aqueous buffer having a pH of about 7.0 to about 7.4.

15. The skin treatment method of claim 14, wherein the water or aqueous buffer is purified and/or sterile.

16. The skin treatment method of claim 13, wherein the at least one cosmetically acceptable excipient is selected from the group consisting of surfactant/emulsifying agents, absorbents, antifoaming agents, binders, biological additives, chelating agents, denaturants, preservatives, solubilizing agents, solvents and thickening agents.

17. The skin treatment method of claim 1, further comprising applying another skin care agent before and/or after the application of the skin care agent and the self heat generation composition.

18. The skin treatment method of claim 17, wherein the other skin care agent is for facial, acne treatment, acne prevention or skin peel.

19. The skin treatment method of claim 1, wherein the self heat generation composition enhances or potentiates the effect of the at least one skin care agent.

20. The skin treatment method of claim 1, wherein the self heat generation composition enhances the dermal absorption of the at least one skin care agent.
